(19) United States
(12) Patent Application Publication
(10) Pub. No.: US 2014/0093975 A1
(43) Pub. Date: Apr. 3, 2014

(54) AUTOMATIC ANALYZER AND SAMPLE ANALYSIS METHOD

(71) Applicant: Shenzhen Mindray Bio-Medical Electronics Co., Ltd., Shenzhen (CN)

(72) Inventors: Hai Wang, Shenzhen (CN); Zhen Zhang, Xuzhou City (CN); Jun Wang, Shenzhen (CN); Peng Zhou, Shenzhen (CN)

(21) Appl. No.: 14/036,764

(22) Filed: Sep. 25, 2013

Related U.S. Application Data
(63) Continuation of application No. PCT/CN2012/072950, filed on Mar. 23, 2012.

(30) Foreign Application Priority Data
Mar. 25, 2011 (CN) 201110074104.3

(51) Int. Cl. G01N 33/53

(52) U.S. Cl. CPC G01N 33/5302
USPC 436/501; 422/69; 422/52

(57) ABSTRACT

An automatic analyzer includes a reaction unit configured for holding a reaction container and carrying the reaction container to a determined operation position, the operation position including a detection operation position; a detection unit configured for detecting analyte in the reaction container of the reaction unit in the detection operation position; a bound-free (“B/F”) unit configured for removing unbound components of a reaction system; and a dispensing unit configured for dispensing reagent and/or a sample to the reaction container, wherein the reaction unit includes an incubation position for incubating a solution in the reaction container.

### TECHNICAL FIELD

This disclosure relates to an automatic analyzer and a sample analysis method.

### BACKGROUND

An immunoassay analyzer is a kind of analyzer with high sensitivity and high specificity. In the clinical context, it is usually used to analyze blood, urine, or other body liquids. There are many techniques used in traditional immunoassay analyzers, e.g., chemiluminescence, electrochemical luminescence, etc.

FIG. 1 illustrates a process of non-homogeneous chemiluminescence immunoassay analysis. When detecting an object substance in a sample, a magnetic bead reagent is formed by coating the magnetic bead with an antibody/antigen. A labeling reagent is formed by marking the antibody with a given marker. There are usually multiple reagent components for analyzing a certain kind of analytical item, e.g., magnetic bead reagent components, marking reagent components, etc. Different components of the reagent can be held in different containers or in different cells of the same reagent container.

During testing, the sample containing an analyte is mixed together with a magnetic bead reagent, a labeling reagent, and other reagents, such that a sample and reagent reaction solution is formed (referred to herein as a “reaction solution”). In certain conditions, reaction complexes are formed by an incubate reaction. Then, using bound-free (“B/F”) technology, unbound markers, other reagents, and the sample of the reaction system are removed. Thereafter, a signal reagent is added, after which the marker of the reaction complexes is reacted with the signal reagent, and generate light. The signal reagent may include one or more types, e.g., luminescent substrate solution, pre-excitation liquid, excitation liquid, and luminescence enhancement solution, etc. There are many techniques for specific coating and B/F, in addition to the magnetic bead method. For example, in another method, the antibody is coated on the wall of the reaction container, plastic beads, etc.

For different characteristics of test items, common immunoassay analyzers are capable of performing different test protocols.

(1) One-Step Protocol

Referring to FIG. 2, a one-step protocol is the simplest protocol. A reagent (may include several kinds of compositions) and a sample are added to a reaction container and mixed together so as to form a reaction solution. Thereafter, the mixed reaction solution is incubated in a thermostatic condition then B/F is performed. A signal reagent is added to the reaction container when B/F is finished, after which optical detection of the reaction container is executed once the signal reagent is added to the reaction container for incubation for a certain time. Certain tests may perform optical detection immediately and do not need incubation after the signal reagent is added. An example includes a chemiluminescence test, which is based on electrochemical luminescence or flash system, as shown in FIG. 3.

(2) Two-Step One B/F Protocol

Referring to FIG. 4, a reagent (called the first reagent, which may include several kinds of compositions) and sample are added to a reaction container and mixed to form a reaction solution. The mixed reaction container is then incubated in a thermostatic condition for a certain time. Thereafter, a reagent (called the second reagent, including several kinds of compositions) is added to the reaction container and mixed with the reaction solution, which is mixed with the first reagent and the sample. Then, the mixed reaction container is incubated in a thermostatic condition for a certain time. The B/F process is executed after incubation, after which the signal reagent is added to the reaction container which B/F is finished. The reaction container to which the signal reagent was added is incubated in a thermostatic condition for a certain time, after which optical detection is executed, as mentioned before. Some kinds of tests may use optical detection directly and do not need incubation after the signal reagent is added.

(3) Two-Step Two B/F Protocol

Referring to FIG. 5, some reagents (called the first reagent, which may include several kinds of compositions) and a sample are added to a reaction container and mixed to form a reaction solution. B/F is executed after the mixed reaction solution is incubated in a thermostatic condition. A reagent (called the second reagent, which may include several kinds of compositions) may be added to the reaction container and mixed with the reaction solution, which is mixed with the first reagent and the sample. The mixed reaction container is incubated in a thermostatic condition for a certain time. B/F is executed after incubation, after which a signal reagent is added to the reaction container which finished the B/F. After the reaction container which added the signal reagent is incubated in a thermostatic condition for a certain time, optical detection is executed, as mentioned before. Some kinds of tests may perform optical detection directly and do not need incubation after the signal reagent is added.

For different testing processes described above, the incubation time is different according to the assay. To obtain accurate test results and support more assays, immunoassay analyzers need to support a very flexible test method.

For traditional immunoassay analyzers, incubation, detection, and B/F functions are set in the same disk, but there are some defects for this design: (1) flexible rotation is restricted because of B/F, so the incubation time is fixed and difficult to adapt to reaction characteristics of different test items; (2) because incubation, detection, and B/F are in the same disk, the disk is too big, and the structure of the disk is complex, so the manufacturing process is difficult; (3) in order to support two steps two B/F protocol, two B/F units are needed, resulting in a high cost; (4) incubation needs to maintain a stable temperature for the disk, but the B/F operation will cause temperature fluctuation in the disk, which causes the unstable results. For other traditional immunoassay analyzers, the incubation of the reaction solution, the B/F, the incubation after the signal reagent is added, and the optical detection is performed separately in different structural units. There are also some defects for this design: (1) the cost is high, the structure is complex, and the size of the analyzer is large; and (2) the energy efficiency is low, and because it needs to coordinate with different units, the control is complex, many test steps are required, and the system structure is complex.

### DISCLOSURE OF THE INVENTION

The present disclosure provides an automatic analyzer and sample analysis method, which can effectively avoid incubation and B/F influencing each other. As a result, the test is more flexible and the structure of the analyzer is simpler.

According to one aspect, an automatic analyzer includes a reaction unit for holding a reaction container and carrying the reaction container to a predetermined operating position, the operating position including a detection operating position; a detection unit for detecting a substance in a reaction container of the reaction unit in a test operating position; a B/F unit for removing unbound components of a reaction system; and a dispensing unit for dispensing reagents and/or samples to the reaction container. An incubation position may be provided for incubating the solution in the reaction container.

According to another aspect, an automatic analyzer includes a reaction unit for holding a reaction container and carrying the reaction container to a predetermined operating position; a detection unit for testing the analyte in the reaction container of the reaction unit in a test operating position; a B/F unit for removing unbound components from the reaction system; and a dispensing unit for dispensing reagent and/or a sample to the reaction container. An incubation position is provided for incubating the solution in the reaction container, the incubation position including the first incubation position and the second incubation position.

According to yet another aspect, a sample analysis method includes a dispensing step for dispensing a sample and a reagent to the reaction container; an incubation step for incubating the reaction container in the reaction unit; a B/F step for removing unbound components from the reaction system; a signal reagent adding step for adding the signal reagent to the reaction container; and a detection step in which the reaction container is carried to a detection operation position by the reaction unit, a detection unit being used to detect the analyte in the reaction container in the detection operation position.

According to still another aspect, a sample analysis method includes a dispensing step for dispensing reagents and/or samples to a reaction container; a first incubation step for a first incubation of the reaction container in the reaction unit; a B/F step for removing unbound components from the reaction system; a signal reagent adding step for adding a signal reagent to the reaction container; a second incubation step for a second incubation of the reaction container in the reaction unit; and a detection step for testing the substance of the reaction container.

In one embodiment, the system separately arranges the reaction unit, which has an incubation function, and B/F unit, which has a clean and separation function, such that the incubation of the reaction container and B/F are finished in different units, preventing incubation and B/F from influencing each other. Accordingly, the automatic analyzer test is more flexible. At the same time, the first incubation, the detection, or the second incubation are all finished in the reaction unit, making the system structure simpler and improving efficiency.

### BRIEF DESCRIPTION OF THE DRAWINGS

FIG. 1 illustrates an immunoassay analyzing process;

FIG. 2 illustrates a one-step protocol;

FIG. 3 illustrates a one-step protocol;

FIG. 4 illustrates a two-step one B/F protocol;

FIG. 5 illustrates a two-step two B/F protocol;

FIG. 6 illustrates a schematic diagram of the first embodiment of an automatic analyzer;

FIG. 7 is a flow chart of a one-step protocol;

FIG. 8 is a flow chart of a two-step one B/F protocol;

FIG. 9 is a flow chart of a two-step two B/F protocol;

FIG. 10 is a schematic diagram of an embodiment of a reaction disk;

FIG. 11 is a schematic diagram of an embodiment of a reaction disk;

FIG. 12 is a schematic diagram of an embodiment of a reaction disk;

FIG. 13 is a schematic diagram of an embodiment of a reaction disk;

FIG. 14 is a schematic diagram of an embodiment of a reaction disk;

FIG. 15 is a schematic diagram of an embodiment of a reaction disk;

FIG. 16 is a schematic diagram of an embodiment of a reaction unit;

FIG. 17 is a schematic diagram of an embodiment of an automatic analyzer;

FIG. 18 is a schematic diagram of an embodiment of an automatic analyzer;

FIG. 19 is a schematic diagram of an embodiment of an automatic analyzer;

FIG. 20 is a schematic diagram of an embodiment of an automatic analyzer; and

FIG. 21 is a schematic diagram of an embodiment of an automatic analyzer.

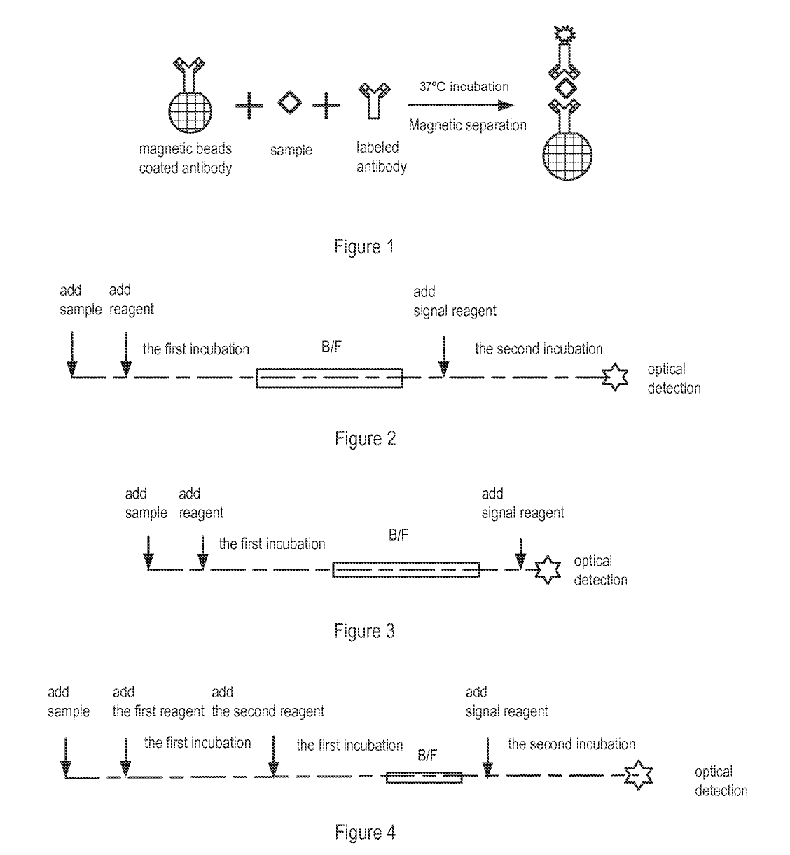

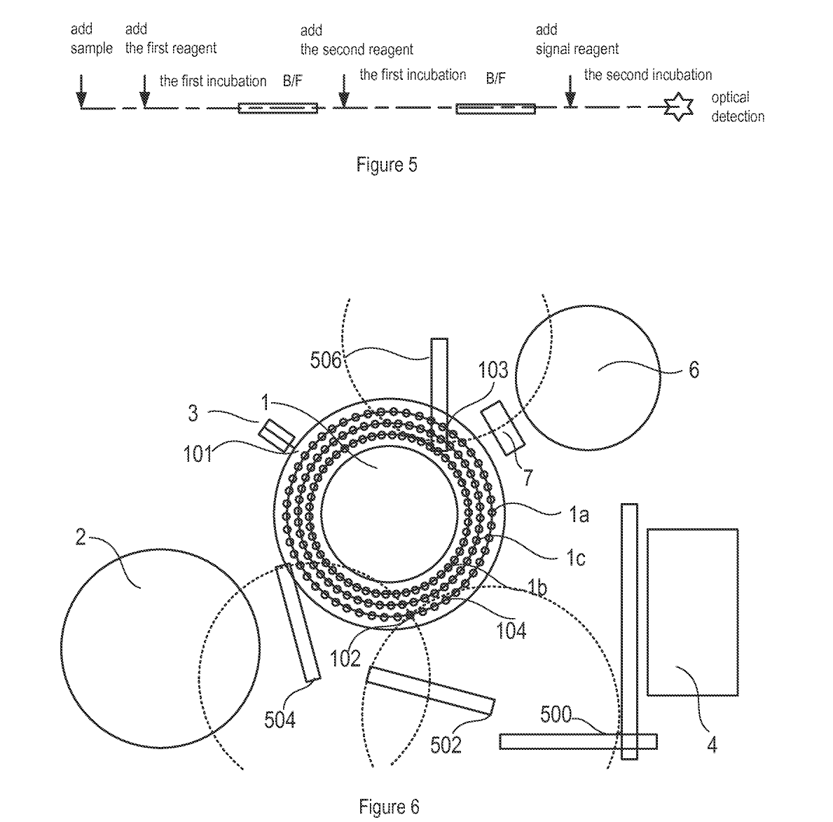

### DETAILED DESCRIPTION

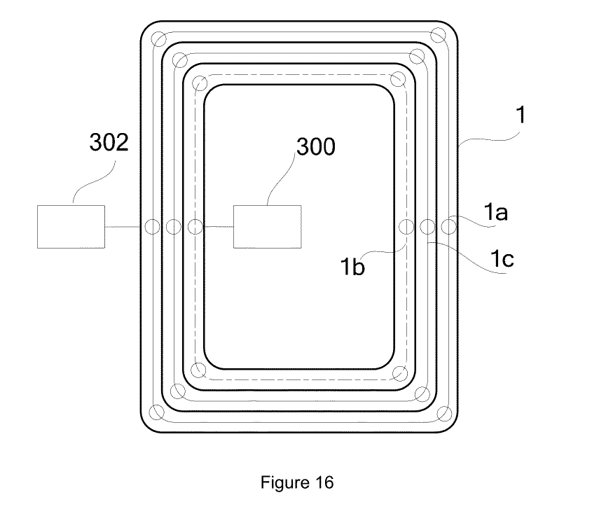

In one embodiment, an automatic analyzer may include a reaction unit for holding a reaction container and carrying the reaction container to a predetermined operating position, the operating position including a detection operating position; a detection unit for detecting a substance in the reaction container of the reaction unit in a test operating position; a B/F unit for removing unbound components from the reaction system; and a dispensing unit for dispensing reagents and/or samples to the reaction container. An incubation position may be provided for incubating a solution in the reaction container. The reaction unit may be circular, as shown in FIG. 6 and FIG. 10-15. It may also be orbital, as shown in FIG. 16. Typically, a reaction disk is used. For simplicity, it is described herein as a circular reaction disk.

The general aim of B/F is removing unnecessary compositions from the reaction system and obtaining a remaining reaction reactant. If magnetic bead coating technology is used, a magnetic field is used for gathering the magnetic beads of the reaction solution on the wall of the reaction container and then removing other residue. If the technology of a reaction container surface coated antibody is adopted, it can clean the reaction container directly.

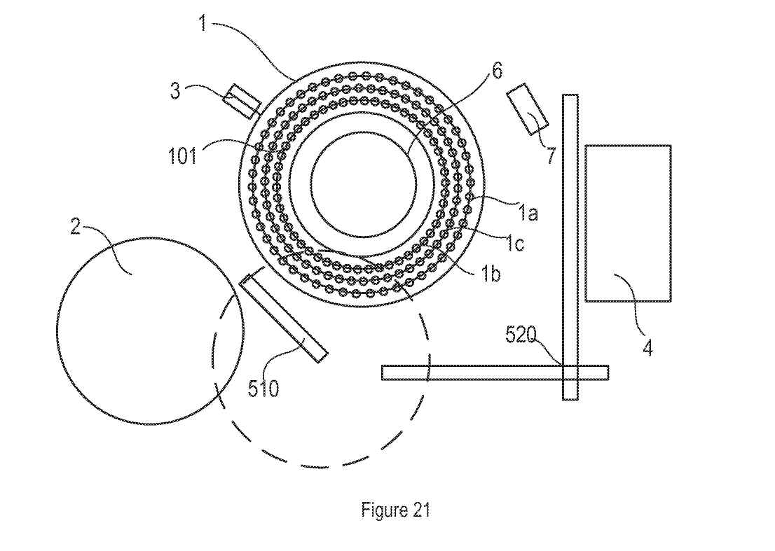

To finish incubation, an automatic analyzer usually controls incubation temperature by a temperature control apparatus. However, the B/F unit may influence the temperature because of the B/F flow. The reaction disk may have an incubation function, but the B/F unit is disposed out of the reaction disk By arranging the incubation unit and B/F unit separately, the incubation temperature is stabilized and the influence of B/F to incubation temperature control is avoided. One skilled in the art may understand that the B/F unit may also be arranged inside the reaction disk, as shown in FIG. 21, which is acceptable as long as there is sufficient spacing of reaction disks to avoid cross-influence between the B/F and reaction disk incubation. Arrangement of the B/F and reaction container can be designed as needed. The reaction container is conveyed between the B/F unit and the reaction disk by a conveyance unit. In another aspect, the incubation of the reaction container and the B/F are separately performed in different units, avoiding cross-influence between the incubation and B/F, making analysis and testing more flexible and improving analyzer efficiency.

FIG. 6 illustrates an embodiment of a one-step protocol. The automatic analyzer may include a reaction disk 1, reagent disk 2, detection unit 3, reaction container supply unit 4, second conveyance unit 500, sample dispensing unit 502, reagent dispensing unit 504, first conveyance unit 506, B/F unit 6, and mix unit 7. The reaction disk may be circular, including at least one ring. Distributed at a number of locations of the reaction disk may be holes or slots, which are used for incubation and/or holding the reaction container, and convey the reaction container to a determined operation position. Operation position is a specific position to which the reaction container of the reaction disk can be conveyed, e.g., the detection operation 101 position of the detection unit 3 for detecting an analyte in the reaction container, dispensing operation position 102 of the dispensing unit for dispensing samples and/or reagents, convey operation positions 103, 104, etc., which are of the conveyance unit for conveying the reaction container.

Those of skill in the art will understand that the operation positions may include more than or less than the operation positions described above. The operation positions may overlap or include other operating positions, for example, operation positions 102 and 104 may be the same position, dispensing operation position 102 may include a sample dispensing operation position and a reagent dispensing position, etc. Reagent disk 2 may be used for containing a reagent container, e.g., reagent bottle. Detecting unit 3 may be used for detecting an analyte in the reaction container in the reaction disk. The detecting unit may include, for example, a photometer for obtaining analyte concentration by detecting light intensity.

Reaction container supply unit 4 may be used to contain a reaction container, e.g., cuvette. The conveyance unit may include the first conveyance unit 506 and the second conveyance unit 500. The second conveyance unit 500 may be used for conveying the reaction container between reaction disk 1 and the reaction container supply unit 4. The dispensing unit may include a sample dispensing unit 502 and a reagent dispensing unit 504. The sample dispensing unit may include a sample needle which is used for drawing and/or discharging a sample. The reagent dispensing unit may be a reagent needle, which is used for drawing and/or discharging a reagent.

In one embodiment, the trajectory of the first conveyance unit intersects with reaction disk 1, B/F unit 6, and pass mix unit 7. The first conveyance unit 506 may be used for conveying the reaction container between the reaction disk 1 and B/F unit 6, and also be used for conveying the reaction container between the reaction disk 1 and the mix unit 7. The B/F unit 6 is used for removing the unbound components of the reaction system. The B/F unit 6 is located outside the reaction disk 1, that is, the B/F unit and reaction disk are arranged independently in one embodiment. The mix unit 7 is used for mixing the reaction solution, although, in some embodiments, certain sample analysis methods do not need mixing.

Reaction container supply unit 4 may supply the reaction container for the automatic analyzer. A new reaction container is conveyed to the convey operation position 104 by the second conveyance unit 500.

In one embodiment, the reaction disk 1, mix unit 7 and B/F unit 6 are located in the orbit of the first conveyance unit 506. The reaction container may be conveyed between the reaction disk 1 and mix unit 7. It also may be conveyed between the reaction disk 1 and B/F unit 6. In some embodiments, two conveyance units may also be set, which convey the reaction container between the reaction disk 1 and B/F unit 6, and convey the reaction container between the reaction disk 1 and mix unit 7.

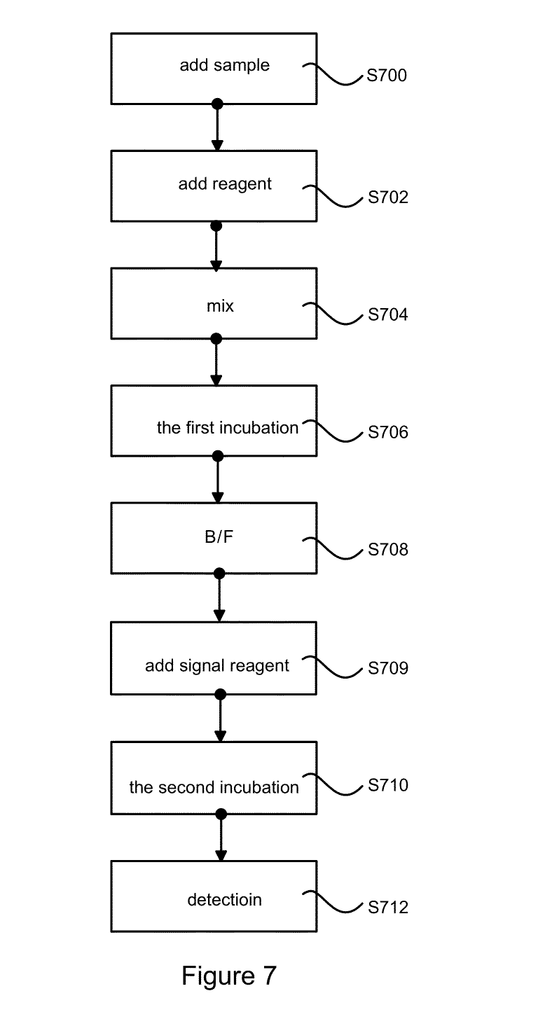

FIGS. 6 and 7 illustrate an exemplary analyzer work flow. Reaction disk 1 may convey a blank position to the operation position 104. The reaction container is conveyed to the reaction disk 1 by the second conveyance unit 500, and then the reaction disk 1 conveys the reaction container to the dispensing operation 102. In S700, the sample is added to the reaction container by the sample dispensing unit 502 in the dispensing operation 102. In S702, the reagent is added to the reaction container by the reagent dispensing unit 504 in the dispensing operation 102. The reaction disk 1 conveys the reaction container to the convey operation position 103. In S704, the first conveyance unit 506 conveys the reaction container. The sample and reagent are then added to the mix unit 7 for mixing. In S706, the reaction container, after mixing is complete, is conveyed to middle ring 1c and/or inner ring 1b of the reaction disk 1 for first incubation. The first incubation position is defined as the position of the reaction disk of the first incubation. In S708, after arriving at the first incubation time, the reaction disk 1 conveys the reaction container back to the convey operation position 103 again, then the reaction container is conveyed to the B/F unit 6 by the first conveyance unit 506.

After several cycles, the reaction container is moved by the B/F unit 6, and then the reaction container is cleaned and separated by the B/F unit 6. There are many kinds of B/F methods, which depend on which coat technology is adopted. For example, in the magnetic B/F method, the antibody or antigen is coated on the surface of magnetic beads. The antibody or antigen can also be coated on the surface of the reaction container, or other solid phase surface, which corresponds to different B/F methods.

In S709, the signal reagent is added to the reaction container. The signal reagent may be added to the reaction container in the B/F unit 6, which can also convey the reaction container to the reaction disk 1 first, and then add the signal reagent to the reaction container, which can also convey the reaction container to a certain position out of the reaction disk 1 and B/F unit 6 first, and then add the signal reagent to the reaction container. For ease of presentation, the disclosure will refer simply to adding the signal reagent to the reaction container in the B/F unit.

Different illumination methods may be used in various embodiments. A method of marker catalyzing signal reagent luminescence may be adopted. A method can also be by the reaction of the marker and signal reagent, or under additional conditions, for example, illuminating by means of an additional electric field, magnetic field, optical excitation, etc., so as to detect the analyte concentration of the sample. For a chemiluminescence analyzer, the general operation flow may include dispensing the reagent and sample, mixing the reaction solution, incubation, B/F, adding a signal reagent, detection, etc. As noted above, some analysis methods do not need mixing.

In S710, after the reaction container with added signal reagent in B/F unit 6 moves to orbit of the first conveyance unit 506 again, the reaction container is conveyed to the outer ring 1a of the reaction disk 1 by the first conveyance unit 506 for incubation, which is after the signal reagent is added. The incubation after adding the signal reagent is defined as the second incubation position. In addition, in certain other testing, incubation after adding the signal reagent is not needed, and the reaction container 1 is conveyed to detection operation position 101 for detecting light, e.g., a chemiluminescence test that is based on electrochemiluminescence, a flash system, etc. In S712, the reaction disk 1 conveys the reaction container to the detection operation position 101. The detection unit 3 detects the analyte in the reaction container, and the detecting signal may be an optical signal of the solution in the reaction container. The reaction container, after detection is finished, is conveyed to the convey operation position 104 by the reaction disk 1. Thereafter, the reaction container is abandoned by the second conveyance unit 500, such that the one-step test is finished.

As will be understood by those skilled in the art, in FIG. 6, the dispensing unit may include a sample dispensing unit 502, a reagent dispensing unit 504, and a conveyance unit (including the first conveyance unit 506 and the second conveyance unit 500). In some implementations, considering the factor of optimal test efficiency, the size of the analyzer and the cost of the dispensing unit and conveyance unit may increase or decrease. The number of dispensing units and conveyance units may be flexibly combined, e.g., just one dispensing unit and just one conveyance unit, or three dispensing units and two conveyance units, etc.

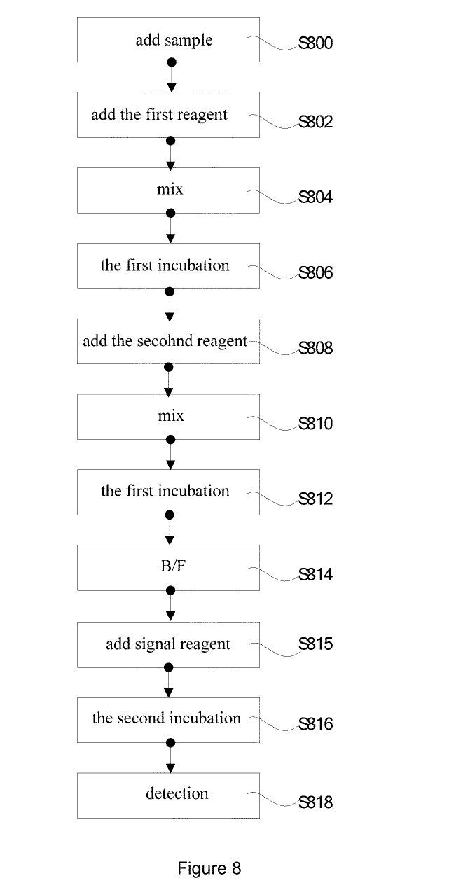

FIG. 8 illustrates another embodiment in which there is only one dispensing unit, which may be a pipetting needle for drawing and discharging samples and reagents. The drawing and discharging of samples and reagents are both finished by dispensing unit 510, as compared with the embodiment of FIG. 6, where a dispensing unit is omitted to save on costs. Referring to FIG. 7, the sample adding step S700 and the reagent adding step S702 are both finished by the dispensing unit 510, other structures and work flow being similar to the embodiments of FIG. 6 and FIG. 7.

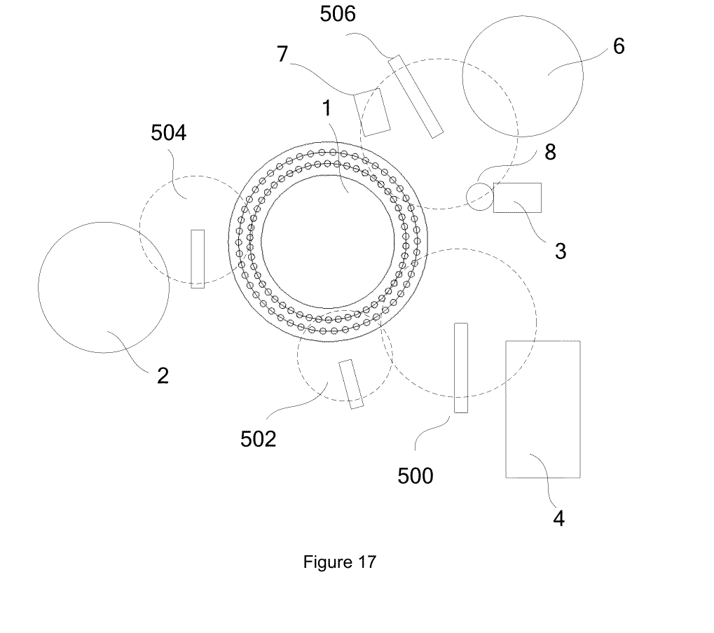

FIG. 17 is another embodiment in which there is just one conveyance unit. Conveyance unit 502 combines the function of the conveying cuvette of the first conveyance unit 506 and the second conveyance unit 500 of FIG. 6, which reduces the cost. The conveyance unit 502 finishes conveying the reaction container between the reaction container supply unit 4, reaction disk 1, B/F unit 6, and mix unit 7 in time, so the reaction container supply unit 4, convey operation positions 103 and 104, B/F unit 6, and mix unit 7 are located in the trajectory of the conveyance unit 506 or intersect with the trajectory of the conveyance unit 506. As shown in the work flow of FIG. 7, the cuvette is conveyed by the conveyance unit 520. Other structures and work flows may be similar to the embodiments of FIG. 6.

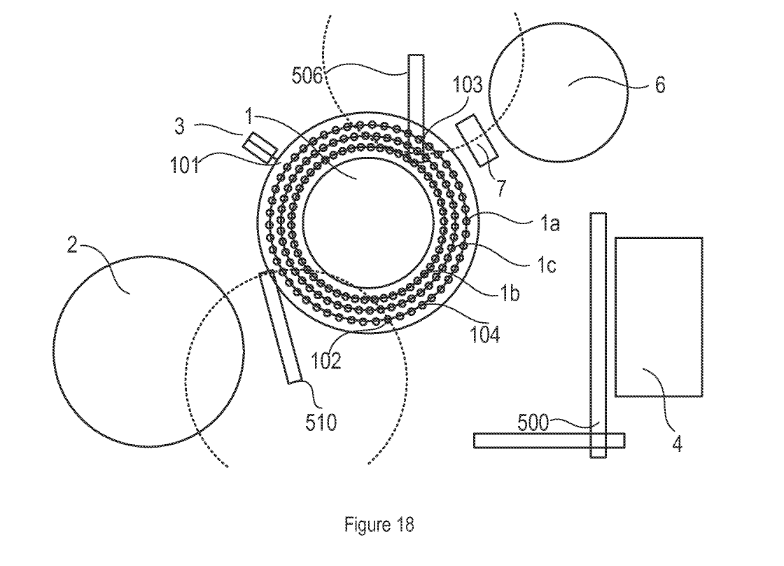

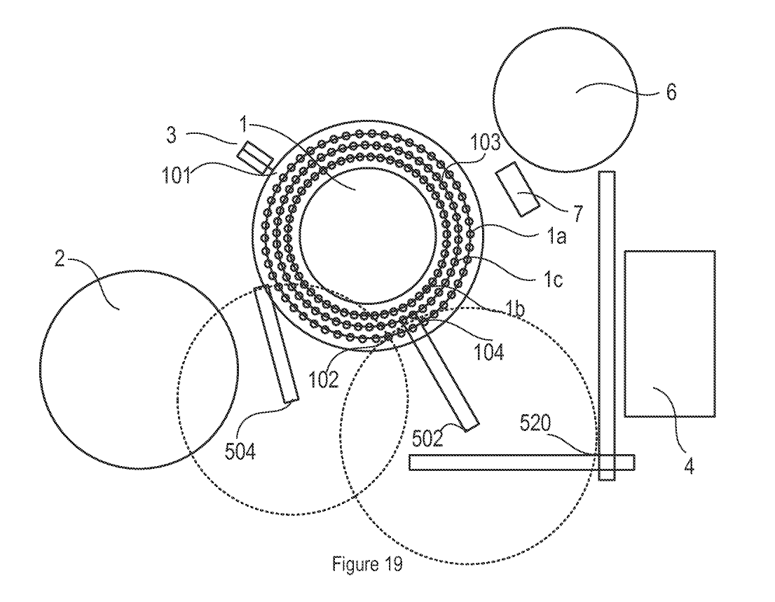

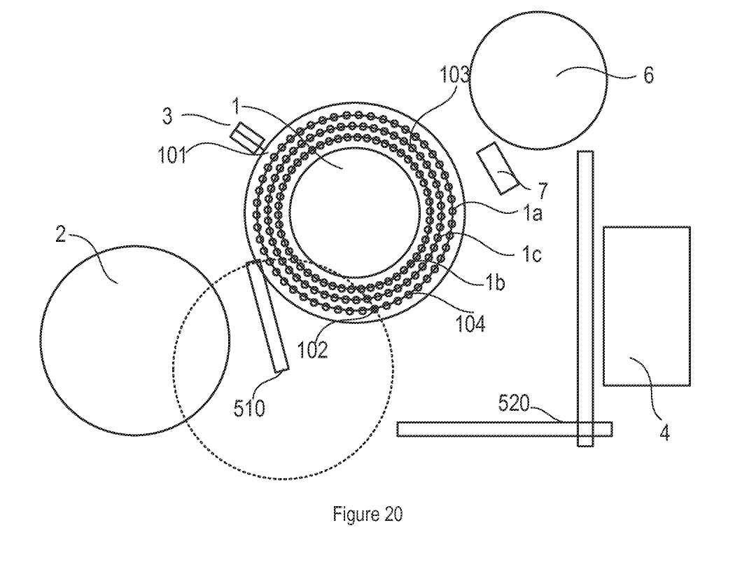

In another embodiment, as shown in FIG. 20, there is just one dispensing unit and just one conveyance unit. The dispensing unit 510 is similar to the dispensing unit of FIG. 18. The conveyance unit 520 is similar to the conveyance unit of FIG. 19. Other structures and work flows may be similar to the embodiments of FIG. 6, which reduce the cost of the system.

In some embodiments of the reaction disk, there is the incubation position, which may be holes or slots. The reaction disk and B/F unit may be structured independently. By arranging the reaction disk and B/F unit separately, it makes incubation and B/F of the reaction container finish in different units, so as to avoid the influence of incubation and B/F. This makes analysis and testing more flexible, while at the same time avoiding the influence of B/F on incubation. Moreover, it allows for control of the temperature and improved work efficiency.

In one embodiment of the disclosed disk, the function of using the optical unit to detect the analyte in the reaction container in the detection operation position, as well as the second incubation of adding a signal reagent, may be integrated in one structure, simplifying the system. After the reaction container to which signal reagent is added has finished the second incubation, the reaction container is turned to the detection operation for detecting the analyte immediately. In this way, the step of conveying the reaction container out of the reaction disk for detection is omitted, reducing test time, simplifying the testing step, and increasing efficiency.

In one embodiment, if there is a multi-ring reaction disk, the detection operation position and the second incubation are in the same ring. When the reaction container finishes the second incubation, it can be turned to the detection operation position to detect. The detection operation position and the second incubation may not be in the same ring. It just needs to convey the reaction container once it has finished the second incubation to the ring which has the detection operation position. The step of conveying the reaction container out of the reaction disk for detecting is also omitted, thus simplifying the detection step and system structure step.

Usually, the temperature conditions of the first incubation and the second incubation in which the signal reagent is added to the reaction container are the same. Therefore, in one embodiment, the first incubation and the second incubation are arranged in the same disk, simplifying the system structure. Because the first incubation and the second incubation are not in a different disk, energy consumption is reduced. For some illumination methods, incubation is not needed after adding the signal reagent. The reaction container can be conveyed to the detection operation position directly.

FIG. 8 illustrates a method for achieving the two-step one B/F protocol. In S800 and S802, the sample and the first reagent are added by the dispensing unit. In S804, the reaction container is conveyed to mix unit 7 for mixing. The mix unit 7 may be independent from the reaction disk 1. The reaction disk 1 can also have a mix position for mixing, and there are many kinds of mix methods, such as ultrasonic wave, etc. In S806, the first incubation for the reaction solution of the sample and reagent is performed. In S808, the reaction container that finished the first incubation is conveyed to the dispensing operation position 102 for adding the second reagent. In S810, the reaction solution after adding the second reagent is mixed. In S812, the first incubation is for the reaction after adding the second reagent. In S814, the B/F of the reaction container is executed. In S815, the signal reagent is added. In step S816, the second incubation is executed. In step S818, detection is performed to detect after finishing the second incubation. Some analysis methods do not need a mixing operation.

As can be seen from the above description, for the automatic analyzer, in reaction disk 1, not only can signal detection of the reaction container in the detection operation position 101 be performed, but for the second incubation for the reaction container of the added signal reagent, the system structure is simplified. After the second incubation following adding the signal reagent, the reaction container may be conveyed to the detection operation position 101 for signal detection, and the step of conveying the reaction container out of the reaction disk for detecting is omitted. Thus, test time is saved and efficiency is increased. In addition, the first incubation and the second incubation after adding the signal reagent for the reaction solution may be arranged in the same ring. Because the first incubation and the second incubation are not in different disks, energy consumption is reduced and the system structure is simplified.

If there are multiple rings on the reaction disk, the first incubation position may be arranged in any ring or any of several rings of the reaction disk 1. The second incubation position may also be arranged in any ring or any of several rings of the reaction disk 1. In one embodiment, the second incubation position may be arranged in the ring that is closest to the detection unit 3. In this way, the reaction container that finished the second incubation may be conveyed by the reaction disk 1 to the detection operation position 101 directly, making the flow easier. As mentioned earlier, some tests do not need incubation after adding the signal reagent. The reaction disk 1 is conveyed to the detection operation position 101 to detect directly.

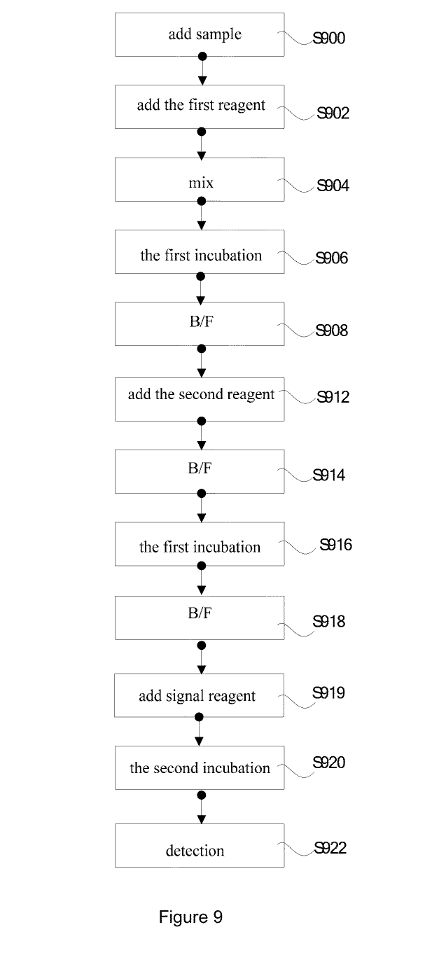

FIG. 9 illustrates a two-step two B/F protocol. In S900 and S902, the dispensing unit adds the sample and the reagent in the reaction container. In S904, the reaction container is conveyed to the mix unit 7 for mixing. In S906, the first incubation of sample and reagent is executed. In S908, the reaction container is conveyed to B/F unit 6 to clean and separate. In S912, the second reagent is added. In S914, the reaction solution is mixed after adding the second reagent. In S916, the first incubation is executed after the solution is mixed. In S918, B/F is performed. In S919, the signal reagent is added. In S920, the second incubation is performed. In S922, detecting is performed. As mentioned earlier, some tests do not need incubation after adding the signal reagent. The reaction disk 1 is conveyed to detection operation position 101 to detect directly. Some analysis methods do not need a mixing operation.

As show in FIG. 6, in order to supply more incubation positions so as to improve the throughput rate, there may be three rings in the reaction disk 1, including an inner ring, a middle ring and an outer ring. In the reaction disk, there are two rings for the first incubation, in which the middle ring and the inner ring may be used for the first incubation. The detection unit 3 is closed to the reaction disk 1 so as to facilitate detection. Because the detection unit 3 is arranged out of the reaction disk 1, the detection operation is realized in the outer ring.

In addition to the embodiment described by way of chemiluminescence, the systems and methods disclosed herein also can apply to an immunoassay analyzer of a fluorescence immunoassay, electrochemiluminescence immunoassay, etc.

In embodiments of the present invention, the technical features or operation step can be combined in any manner. Those having skill in the art will understand that the sequence of the steps or actions can be changed in various embodiments. Accordingly, unless otherwise stated, there is no requirement of a certain order.

In various embodiments, steps may be embodied as executable instructions that may be performed by a general-purpose or special-purpose computer (or other electronic device). These steps may be performed by hardware components, which include specific logic circuits for performing these steps, or performed by any suitable combination of hardware, software and/or firmware.

As described above, these three methods all include the second incubation after adding the signal reagent. However, some tests do not need incubation after adding the signal reagent. The aforementioned methods may be extended, for example, by adding the third reagent, etc. The reaction container, after completion of the second incubation, does not need be conveyed out of the reaction disk 1 for detection in the detection position. Therefore, the system is simplified and the test time is reduced. In some embodiments, the reaction container can be conveyed to the detection operation position 101. The reaction disk includes the first incubation position for the reaction solution. When incubation after adding the signal reagent is needed, the reaction disk may also include the second incubation position for the reaction container to which is added the signal reagent.

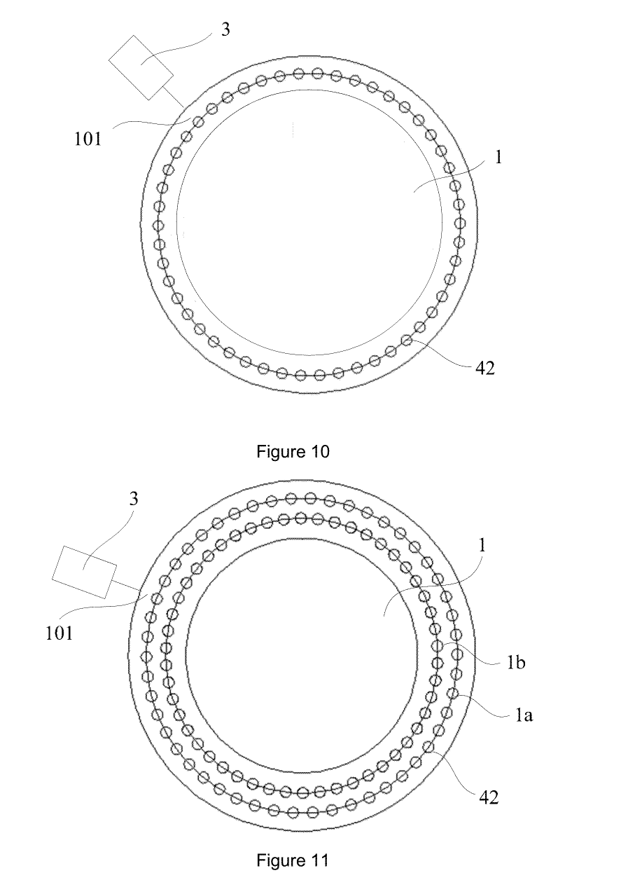

Referring to FIG. 10, there may be only one ring in the reaction disk 1 on which is located a number of positions. These positions may be holes, slots, etc., for incubation and/or holding the reaction container 42 and conveying the reaction container 42 to a determined operation position. The operation position may include a detection operation position 101. The detection unit 3 is out of the reaction disk 1, i.e., out of the rings of the reaction disk. When the reaction container 42 is turned to the detection operation position 101, the detection unit 3 detects and analyzes the signal of the reaction container 42. The reaction disk 1 also includes the first incubation position, which is for incubation of the reaction solution. The reaction disk 1 may also include the second incubation position, which is for incubation of the reaction container 42. The detection unit 3 may be located within the reaction container 1. Similarly, the reaction container 42 is detected and analyzed when it is turned to the detection operation position 101.

Because a plurality of functions are implemented in one reaction disk, the system is simplified, the test time is reduced, the test flow is simplified, and the efficiency of the analysis of the automatic analyzer is improved. The incubation position and B/F position may be located separately. The mutual influence of B/F and incubation is thus avoided. It is easier to control the temperature of the reaction disk and the operation of B/F, improving the efficiency of incubation and B/F.

In other embodiments, the reaction disk may include a set of two rings. The reaction disk is turned to a specific operating position, which includes a detection operating position. The detection unit is out of the reaction disk. When incubation after adding the signal reagent is needed, the outer ring may include the second incubation position and the inner ring may include the first incubation position.

If there is blank position in the outer ring, the outer ring can also perform the first incubation. The detection unit may also be located within the reaction container. Then, by locating the outer ring including the first incubation position, the inner ring may include the second incubation position. If there is a blanket position in the inner ring, the inner ring may also perform the first incubation. Based on the above embodiments, the reaction container may arrange multiple rings, e.g., three rings or more, such that more reaction containers can be held or incubated.

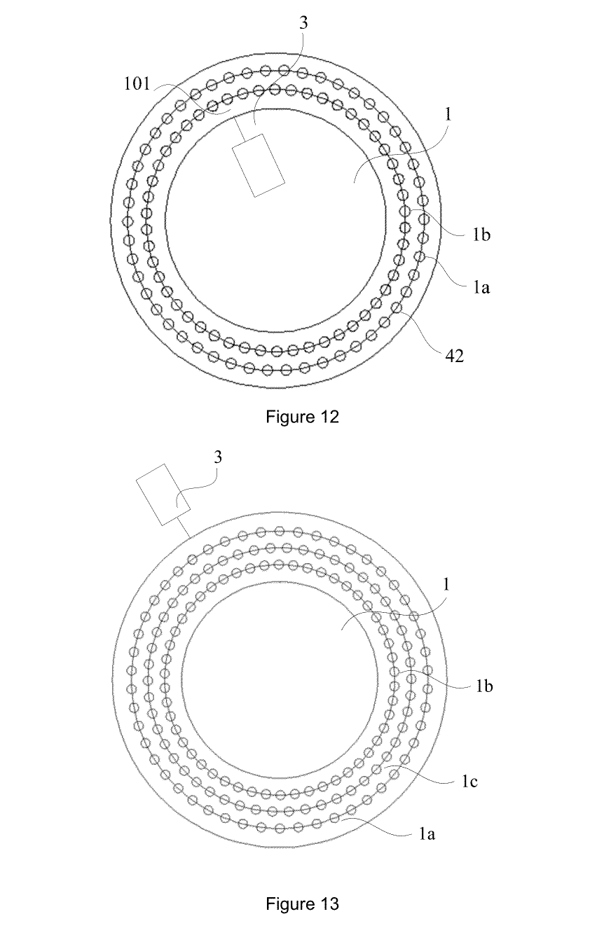

Referring to FIG. 11, in one embodiment, there are two rings in the reaction disk 1, including outer ring 1a and inner ring 1b. The reaction disk 1 is rotated to a specific operating position, which includes a detection operating position 101. The detection unit 3 is out of the reaction disk 1. When incubation after adding the signal reagent is needed, the outer ring 1a may include the second incubation position and the inner ring 1b may include the first incubation position. The detection unit 3 may also be located at the inner of the reaction disk 1, as shown in FIG. 12. The outer ring 1a includes the first incubation position. When incubation after adding the signal reagent is needed, the inner ring 1b may also include the second incubation position.

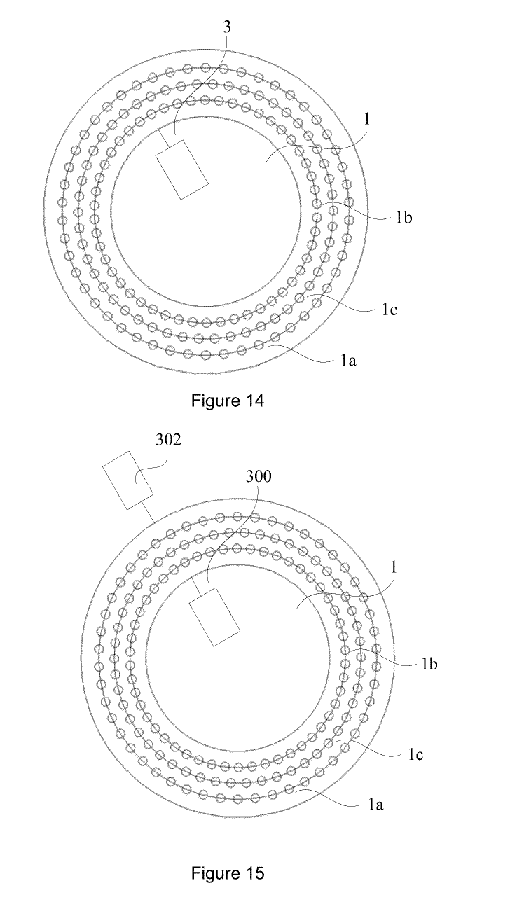

Referring to FIG. 13, there are three rings in the reaction disk 1, including an outer ring 1a, an inner ring 1b, and a middle ring 1c. The reaction disk 1 is turned to a specific operating position, which may include a detection operating position. The detection unit 3 is out of the reaction disk 1. When incubation after adding the signal reagent is needed, the outer ring 1a may include the second incubation position and the inner ring 1b and the middle ring 1c may include the first incubation position. In this manner, the number of incubation positions is increased. At the same time, the reaction disk 1 may hold more reaction containers for incubation, so the test efficiency is increased. The detection unit 3 may also be located at the inner of the reaction disk 1.

As shown in FIG. 14, the outer ring 1a and the middle ring 1c include the first incubation position. When incubation after adding the signal reagent is needed, the inner ring 1b may also include the second incubation position. Thus, the number of incubation positions is increased, as the reaction disk 1 may hold more reaction containers for incubation, increasing test efficiency. The detection unit 3 may be arranged both at the outer and inner portions of reaction disk 1.

Referring to FIG. 15, the reaction disk 1 is rotated to a specific operating position. Two detection units are arranged, including outer detection unit 302 and inner detection unit 300. The outer detection unit 302 is outer of the reaction disk 1, and the inner detection unit 300 is inner of reaction disk 1. When incubation after adding the signal reagent is needed, the outer ring 1a and the inner ring 1b may also include the second incubation position and the middle ring 1c may include the first incubation position. Further, by increasing the rings of the reaction container, more reaction containers are available to hold to incubation and detection.

The system may include an analyzer, including: a reaction unit, for holding the reaction container and carrying the reaction container to a determined operation; a position detection unit, for detecting the analyte in the reaction container; a B/F unit, for removing the unbound components of the reaction system; and a dispensing unit, for dispensing the reagent and/or sample to the reaction container. There is an incubation position for incubating the solution in the reaction container in the reaction unit.

By arranging the incubation and B/F separately, it avoids the influence of the B/F operation on incubation temperature. By avoiding the restriction of the B/F operation to the incubation operation, the incubation efficiency is improved. At the same time, it makes the test flow more flexible, because the incubation unit includes the first incubation position and the second incubation position. The two incubations are both incubated in the reaction disk, improving incubation efficiency, saving energy, and simplifying the system structure.

Referring to FIG. 17, the automatic analyzer may include a reaction disk 1, a reagent disk 2, a detection unit 3, a reaction container supply unit 4, a second conveyance unit 500, a sample dispensing unit 502, a reagent dispensing unit 504, a first conveyance unit 506, a B/F unit 6, and a mix unit 7. The detection unit 3 of the automatic analyzer includes detection position 8.

In one embodiment, the reaction disk is a ring structure, include at least one ring. There are a number of positions in the ring. These position may be slots, holes, etc., for incubation and/or holding the reaction container to a determined operation. Other units do the corresponding operations, e.g., adding a sample or a reagent, or conveying. The reaction disk 1 includes an incubation position, including the first incubation position and the second incubation position. The reagent container 2 is used for containing the reagent container, for example, the reagent bottle. The detection position 8 is independent from the reaction disk 1. It may be arranged out of the reaction disk 1, and it may also be arranged within reaction disk 1. The detection unit 3 is used for detecting the analyte in the reaction container which is in detection position 7. The detection unit 3, e.g., photometer, detects the optical signal to test the content of the analyte. The reaction container supply unit 4 is used for containing the reaction container, e.g., a cuvette. The second conveyance unit 500 is used for conveying the reaction container between the reaction disk 1 and the reaction container supply unit 4. The dispensing unit includes a sample dispensing unit 502, which is used for dispensing a sample, and the reagent dispensing unit 504. The sample dispensing unit 502 may be a sample needle, which is used for drawing and discharging the sample. The reagent dispensing unit 504 may be a reagent needle, which is used for drawing and discharging the reagent. The reaction disk 1, mix unit 7, B/F unit 6, and detection unit 3 are in the trajectory of the first conveyance unit 506, which is used for conveying the reaction container between the reaction disk 1 and the B/F unit 6. It also may be used for conveying the reaction container between the reaction disk 1 and the mix unit 7, and/or for conveying the reaction container between the reaction disk 1 and the detection position 8. The B/F unit 6 is used to remove the unbound components from the reaction system. The mix unit 7 is used to mix the reaction solution. However, some analysis methods do not need mixing.

During analysis, the reaction container is conveyed from the reaction disk 1 to the detection unit 3, then by the detection unit 3 to detect the analyte in the reaction container, which is in the detection position 8. Because the optical signal of the solution in the reaction container is very low, it is easily be disturbed by outside influences. The optical signal detection needs a dark environment, which is easier to form in structure by setting the detection position 8 individually. In another aspect, the restriction of detection to incubation and conveying is avoided. Because the second incubation which is after adding signal reagent is finished in the reaction disk 1, the detection unit 3 does not need to provide the second incubation. In another embodiment, the detection position 8 is not arranged individually. However, detecting the sample in the reaction disk 1, that is, the reaction container of the reaction disk, is detected in the detection operation position 101 by the detection unit, which is similar to FIG. 6.

In a specific test, adding a sample and reagent in the reaction disk 1, adding a sample by the sample dispensing unit 502, and adding reagent by the reagent dispensing unit 504, as described before, it can also add a reagent first, then add a sample. The reaction container is conveyed to the mix unit 7 for mixing by the first conveyance unit 506. The reaction container is conveyed to the first incubation position of the reaction disk 1 for the first incubation by the first conveyance unit 506. When the first incubation is finished, the reaction container is conveyed to the B/F unit 6 to clean and separate by the first conveyance unit 506, and the signal reagent is added; as mentioned earlier, the signal reagent can be added to the other position. Then, the reaction container is conveyed to the second incubation position of the reaction disk 1 for the second incubation by the first conveyance unit 506. When the second incubation is finished, the reaction container is conveyed to the detection unit 3 for detecting by the first conveyance unit 506.

The reaction disk may be embodied as a ring structure, including at least one ring, which includes the first incubation position and the second incubation position. Two detection units 3 may also be provided. Then, the reaction container can be used with optical detection, increasing the numbers of detection and improving the analysis efficiency of analyzer. The mix unit 7 may be arranged individually and also may be arranged in the reaction disk 1. The detection unit 3 may be arranged individually as the previous embodiments, and also may be set around the reaction disk 1. The reaction container is detected in the detection operation position 101 by the detection unit 3.

The present disclosure also provides a sample analysis method, including: a dispensing step for adding the sample and reagent to the reaction container, an incubation step for incubating the reaction container in the reaction unit, an adding signal reagent step for adding signal reagent in the reaction container, and a detection step where the reaction container is conveyed to the detection operation by the reaction unit and the analyte in the reaction container in the detection operation position is detected by the detection unit.

The incubation step is executed in the reaction disk. The B/F is executed in the B/F unit. In one embodiment, the incubation step and the B/F step are executed in different structures, avoiding the influence between the B/F step and the incubation step, which controls the temperature condition of incubation, thus improving work efficiency. Not only is the incubation step executed in the reaction disk, but the detection step is also executed in the detection operation position by the detection unit. By combining many functions together, the system structure is simplified. The reaction container does not need to be conveyed to the detection position, which is out of the reaction disk for detection and analysis. The operation which conveys the reaction container when it is finished with the second incubation out of the reaction disk for detection is omitted, reducing test time, simplifying the test step, and improving the efficiency of the automatic analyzer. In one embodiment, the first incubation step and the second incubation step are executed in the same disk. Thus, the system structure is simplified, and because the incubations do not need to be in two different disks, energy is saved. In some illumination methods, incubation is not needed after adding the signal reagent.

The present disclosure also provides a sample analysis method, including, adding sample and reagent to the reaction container, the first incubation step for incubating the reaction container in the reaction unit, a B/F step for removing unbound components of the reaction system, an adding signal reagent step for adding the signal reagent to the reaction container, a second incubation step for the second incubation to the reaction container in the reaction unit, and the detection step for detecting analyte in the reaction container.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An automatic analyzer, including:
a reaction unit configured for holding a reaction container and carrying the reaction container to a determined operation position, the operation position including a detection operation position;
a detection unit configured for detecting analyte in the reaction container of the reaction unit in the detection operation position;
a bound-free (“B/F”) unit configured for removing unbound components of a reaction system; and
a dispensing unit configured for dispensing reagent and/or a sample to the reaction container;
wherein the reaction unit includes an incubation position for incubating a solution in the reaction container.

2. The analyzer of claim 1, wherein the automatic analyzer includes a conveyance unit configured for conveying the reaction container between the reaction unit and the B/F unit.

3. The analyzer of claim 1, wherein the incubation position includes a first incubation position and a second incubation position.

4. The analyzer of claim 3, wherein the reaction unit is a reaction disk, the reaction disk includes at least two rings, the detection unit is arranged outside and/or inside of the reaction disk, and the outer ring and/or inner ring of the reaction disk include the second incubation position.

5. The analyzer of claim 3, wherein the reaction unit is a reaction disk; the reaction disk includes at least three rings including an outer, middle, and inner ring; the outer ring includes the second incubation position; the inner ring and the middle ring include the first incubation position; and the detection unit is arranged outside of the reaction disk.

6. The analyzer of claim 1, further comprising a mix unit configured for mixing solution in the reaction container.

7. The analyzer of claim 1, further comprising a reaction container supply unit configured for containing a reaction container supply.

8. The analyzer of claim 7, further comprising a conveyance unit configured for conveying the reaction container between the reaction unit, the B/F unit, and the reaction container supply unit.

9. The analyzer of claim 8, wherein the conveyance unit includes a first conveyance unit and a second conveyance unit, the first conveyance unit for conveying the reaction container between the reaction unit and the B/F unit, and the second conveyance unit for conveying the reaction container between the reaction unit and the reaction container supply unit.

10. The analyzer of claim 1, wherein the dispensing unit includes a sample dispensing unit and a reagent dispensing unit, the sample dispensing unit configured for dispensing sample to the reaction container and the reagent unit configured for dispensing reagent to the reaction container.

11. The analyzer of claim 1, wherein the dispensing unit is a single dispensing unit configured for dispensing a sample and reagent to the reaction container.

12. An automatic analyzer, including:
a reaction unit configured for holding a reaction container and carrying the reaction container to a determined operation position;
a detection unit configured for detecting analyte in the reaction container of the reaction unit;
a B/F unit configured for removing unbound components of a reaction system; and
a dispensing unit configured for dispensing reagent and/or a sample to the reaction container;
wherein the reaction unit includes an incubation position configured for incubating solution in the reaction container, the incubation position including a first incubation position and a second incubation position.

13. The analyzer of claim 12, further comprising a conveyance unit configured for conveying the reaction container between the reaction unit and the B/F unit.

14. The analyzer of claim 12, wherein the reaction unit is a reaction disk, the reaction disk includes at least two rings, at least one ring includes the first incubation position, and at least one ring includes the second incubation position.

15. The analyzer of claim 12, further comprising a mix unit configured for mixing solution in the reaction container.

16. The analyzer of claim 12, further comprising a reaction container supply unit for containing a reaction container supply.

17. The analyzer of claim 16, further comprising a conveyance unit for conveying the reaction container between the reaction unit, the B/F unit, and the reaction container supply unit.

18. The analyzer of claim 17, wherein the conveyance unit includes a first conveyance unit and a second conveyance unit, the first conveyance unit configured for conveying the reaction container between the reaction unit and the B/F unit, and the second conveyance unit configured for conveying the reaction container between the reaction unit and the reaction container supply unit.

19. The analyzer of claim 12, wherein the dispensing unit includes a sample dispensing unit and a reagent dispensing unit, the sample dispensing unit configured for dispensing sample to the reaction container, and the reagent unit configured for dispensing reagent to the reaction container.

20. The analyzer of claim 12, further comprising a detection position independent of the reaction unit, the detection unit for detecting the analyte in the reaction container at the detection operation position.

21. A sample analysis method, including:
dispensing a sample and reagent to a reaction container;
incubating the reaction container in a reaction unit;
performing a bound-free (“B/F”) process for removing unbound components of a reaction system;
adding a signal reagent to the reaction container;
carrying the reaction container to a detection operation position by the reaction unit; and
detecting an analyte in the reaction container in the detection operation position by a detection unit.

22. The method of claim 21, further including:
conveying the reaction container between the reaction unit and a B/F unit for performing the B/F process.

23. The method of claim 21, further including:
mixing a solution in the reaction container.

24. The method of claim 21, including:
conveying the reaction container to the reaction unit by a conveyance unit.

25. The method of claim 21, including:
adding a signal reagent to the reaction container in a B/F unit.

26. A sample analysis method, including:
a dispensing step for dispensing a sample and a reagent to reaction container;
a first incubation step for incubating the reaction container in a reaction unit;
a bound-free (“B/F”) step for removing unbound components of a reaction system;
an adding signal reagent step for adding signal reagent to the reaction container;
a second incubation step for incubating the reaction container in the reaction unit; and
a detection step for detecting an analyte in the reaction container.

27. The method of claim 26, further comprising
conveying the reaction container between the reaction unit and a B/F unit.

28. The method of claim 26, further comprising:
conveying the reaction container between the reaction unit and a detection position which is independent from the reaction unit.
